(19) United States
(12) Patent Application Publication
(10) Pub. No.: US 2008/0243395 A1
(43) Pub. Date: Oct. 2, 2008

(54) IMAGE DIAGNOSIS SUPPORTING APPARATUS AND SYSTEM

(75) Inventors: Akira Oosawa, Tokyo (JP); Takayuki Udagawa, Kawasaki-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/058,733

(22) Filed: Mar. 30, 2008

(30) Foreign Application Priority Data
Mar. 30, 2007 (JP) 2007-095442

(51) Int. Cl. G01N 33/48

(52) U.S. Cl. 702/19

(57) ABSTRACT

In a disease case DB, disease case images are classified by disorder and registered. In a feature quantity DB, a feature quantity (second feature quantity) of each disease case image classified by disorder is registered. A server compares a feature quantity (first feature quantity) of a lesion site included in a diagnosis target image, with the second feature quantity on a disorder-by-disorder basis, retrieves representative disease case images based on a comparison result from the disease case DB on a disorder-by-disorder basis, and provides a user with retrieved representative disease case images. Therefore, the user can obtain disease case images useful for determining a disorder or statistical information and the like regarding the disorder during image diagnosis based on the diagnosis target image.

### BACKGROUND OF THE INVENTION

1. Field of the Invention

The present invention relates to an image diagnosis supporting apparatus and system, and in particular, to a technique for retrieving and presenting, based on feature quantities extracted from a diagnosis target image, disease case images similar to the diagnosis target image.

2. Description of the Related Art

Conventionally, a diagnosis supporting system is proposed which takes text information of a new disease case as a query to retrieve disease cases having high degrees of credibility (degrees of similarity) from a database containing previous disease cases, further classifies the retrieved similar disease cases according to disorder and calculates a degree of similarity for each disorder, and displays the names of diseases having high degrees of similarity and disorder information thereof on a screen (Japanese Patent Application Laid-Open No. 2004-288047).

In addition, a diagnosis supporting apparatus is proposed which collates a feature quantity detected from a lesion site of a diagnosed image with feature quantities of reference images accumulated in a database, calculates degrees of similarity of the reference images with respect to the diagnosed image, and retrieves the reference images in the order of the degrees of similarity (Japanese Patent Application Laid-Open No. 2002-230518). Moreover, in Japanese Patent Application Laid-Open No. 2002-230518, there is disclosed a technique for computing a probability for each disease name of reference images and displaying disease names in the order of probability (refer to FIG. 10 in Japanese Patent Application Laid-Open No. 2002-230518).

The diagnosis supporting system described in Japanese Patent Application Laid-Open No. 2004-288047 does not support image diagnosis and, therefore, is incapable of retrieving a disease case image using image information as a query. In addition, since determination is solely based on degrees of similarity and does not consider incidence, frequently occurring disorders are mixed with rare disorders, creating a problem in that information useful for diagnostics cannot be provided in an efficient manner.

Meanwhile, since the diagnostic supporting apparatus described in Japanese Patent Application Laid-Open No. 2002-230518 is configured so that reference images are to be retrieved in order of degree of similarity, if a large number of reference images of a disorder similar to the diagnosed image is registered in the database, there is a risk that reference images of other disorders with low degrees of similarity will not be retrieved. Furthermore, Japanese Patent Application Laid-Open No. 2002-230518 fails to describe both a method for displaying reference images (disease case images) and a method for processing statistical information regarding disease cases and disease cases with a small number of samples.

### SUMMARY OF THE INVENTION

The present invention has been made in consideration of the above circumstances, and an object thereof is to provide an image diagnosis supporting apparatus and system capable of presenting disease case images useful for determining a disorder and statistical information and the like regarding the disorder when performing image diagnosis based on a diagnosis target image.

In order to achieve the above-described object, an image diagnosis supporting apparatus according to a first aspect of the present invention comprises: a feature quantity calculating device which calculates, based on a diagnosis target image, a first feature quantity of a lesion site included in the diagnosis target image; a database in which a plurality of disease case images and second feature quantities of lesion sites in the respective disease case images are classified by disorder and registered in association with each other; a retrieving device which compares the first feature quantity with the second feature quantities on a disorder-by-disorder basis and retrieves a disease case image based on a comparison result on a disorder-by-disorder basis from the database; and a displaying device which displays the disease case image retrieved by the retrieving device on a disorder-by-disorder basis.

The invention according to the first aspect enables a disease case image which is very similar to a diagnosis target image retrieved for each disorder (representative disease case image) to be presented, and thus, prevents presented disease case images from being limited to particular disorders.

According to a second aspect of the present invention, the image diagnosis supporting apparatus according to the first aspect further comprises: a determining device which determines whether a disorder requiring attention when reference thereof is included in the disease case images retrieved from the database; and an alarming device which causes, when the determining device determines that the disorder requiring attention is included, a warning alerting that the disorder requiring attention is included to be displayed on the display device. In other words, in the case where a disorder requiring special attention is retrieved, the image diagnosis supporting apparatus is arranged so as to invite attention when the user references a disease case image of the disorder.

According to a third aspect of the present invention, in the image diagnosis supporting apparatus according to the second aspect, the disorder requiring attention is a disorder for which a small number of samples is contained in the database. Regarding a disorder with a small number of samples, large variation occurs in degrees of similarity which are calculated based on the first and second feature quantities. Therefore, the reliability of degrees of similarity with respect to a diagnosis target image is low. Thus, the image diagnosis supporting apparatus is arranged so as to invite attention when referencing such a disorder.

According to a fourth aspect of the present invention, in the image diagnosis supporting apparatus according to the second or third aspect, the alarming device causes at least one of disorder information regarding the disorder requiring attention and disorder information regarding confusable disorders to be displayed on the display device in association with the display of the warning. In other words, the image diagnosis supporting apparatus is arranged so as to present disorder information (for example, typical findings, typical disease case images and the like) of a disorder requiring attention and to present disorder information on disorders likely to be confused with the disorder requiring attention.

According to a fifth aspect of the present invention, in the image diagnosis supporting apparatus according to any of the first to fourth aspects: statistical information regarding disorders is registered into the database on a disorder-by-disorder basis; the retrieving device retrieves disease case images and statistical information on a disorder-by-disorder basis from the database; and the display device displays the disease case images and the statistical information retrieved by the retrieving device.

By referencing statistical information presented on a disorder-by-disorder basis, image diagnostics can be performed while determining whether a disease case is a frequently occurring disease case or a rare disease case by giving due consideration to variations in the degrees of similarity or the like.

According to a sixth aspect of the present invention, in the image diagnosis supporting apparatus according to any of the first to fifth aspects, the disease case images classified by disorder are further classified into sub-classifications within each disorder.

According to a seventh aspect of the present invention, in the image diagnosis supporting apparatus according to the sixth aspect, the retrieving device compares the first feature quantity and the second feature quantities on a disorder-by-disorder basis, and retrieves a disease case image based on a comparison result on a disorder-by-disorder basis from the database and on a sub-classification by sub-classification basis.

Even with a particular disorder, in the case where large variation exists in feature quantities (second feature quantities) according to degree of progression of the disease or the like and where such second feature quantities can be further classified (subclassified), the image diagnosis supporting apparatus is arranged to retrieve similar disease case images (representative disease case images) on a sub-classification by sub-classification basis.

An eighth aspect of the present invention is an image diagnosis supporting system in which a user terminal and a disease case information server are connected to each other via a network, wherein the user terminal comprises: a feature quantity calculating device which calculates, based on a diagnosis target image, a first feature quantity of a lesion site included in the diagnosis target image; a first communication device which transmits the calculated first feature quantity to the disease case information server and receives a disease case image retrieved by the disease case information server; and a display device which displays the received disease case image, whereas the disease case information server comprises: a database in which a plurality of disease case images and second feature quantities of lesion sites in the respective disease case images are classified by disorder and registered in association with each other; a retrieving device which compares the first feature quantity with the second feature quantities on a disorder-by-disorder basis and retrieves a disease case image based on a comparison result on a disorder-by-disorder basis from the database; and a second communication device which receives the calculated first feature quantity from the user terminal and transmits the disease case image retrieved on a disorder-by-disorder basis to the user terminal.

A ninth aspect of the present invention is an image diagnosis supporting system in which a user terminal and a disease case information server are connected to each other via a network, wherein the user terminal comprises: a first communication device which transmits a diagnosis target image to the disease case information server and which receives a disease case image retrieved by the disease case information server; and a display device which displays the received disease case image, whereas the disease case information server comprises: a feature quantity calculating device which calculates, based on the diagnosis target image, a first feature quantity of a lesion site included in the diagnosis target image; a database in which a plurality of disease case images and second feature quantities of lesion sites in the respective disease case images are classified by disorder and registered in association with each other; a retrieving device which compares the first feature quantity with the second feature quantities on a disorder-by-disorder basis and retrieves a disease case image based on a comparison result on a disorder-by-disorder basis from the database; and a second communication device which receives the diagnosis target image from the user terminal and transmits the disease case image retrieved on a disorder-by-disorder basis to the user terminal.

According to a tenth aspect of the present invention, in image diagnosis supporting system according to the eighth or ninth aspect, the disease case information server further comprises: a determining device which determines whether a disorder requiring attention when reference thereof is included in the disease case images retrieved from the database; and an alarming device which causes, when the determining device determines that the disorder requiring attention is included, a warning alerting that the disorder requiring attention is included to be displayed on the display device of the user terminal.

According to an eleventh aspect of the present invention, in the image diagnosis supporting system according to the tenth aspect, the alarming device causes at least one of disorder information regarding the disorder requiring attention and disorder information regarding confusable disorders to be displayed on the display device in association with the display of the warning.

According to a twelfth aspect of the present invention, in the image diagnosis supporting system according to any of the eighth to eleventh aspects: statistical information regarding disorders is registered into the database on a disorder-by-disorder basis; the retrieving device retrieves disease case images and statistical information on a disorder-by-disorder basis from the database; and the second communication device transmits the disease case images and the statistical information retrieved by the retrieving device to the user terminal.

According to the aspects of the present invention, disease case images similar to a diagnosis target image can be retrieved and representative images can be displayed on a disorder-by-disorder basis. In addition, by inviting attention in the case where a disease case image of a disorder requiring attention during reference thereof such as a disease case with a small number of samples is retrieved and by further presenting statistical information of each disorder, it is possible to prevent oversights and erroneous diagnoses.

### BRIEF DESCRIPTION OF THE DRAWINGS

FIG. 1 is a system configuration diagram showing an image diagnosis supporting system according to an embodiment of the present invention;

FIG. 2 is a flowchart showing processing performed on a user terminal side;

FIG. 3 is a flowchart showing processing performed on a disease case information server side;

FIG. 4 is a table showing a data example of first feature quantities extracted from a query image;

FIG. 5 is a table showing data examples of second feature quantities of respective disease case images stored in a feature quantity DB and classified according to disorder;

FIG. 6A shows an example of display of a graph plotting a relationship between respective disease case images of a disorder A and a query image;

FIG. 6B shows an example of display of a result obtained by sorting respective retrieved disease cases of the disorder A according to degrees of similarity and then clustering according to sub-classifications;

FIG. 6C shows an example of display of disease case information;

FIG. 6D shows an example of display of diagnosis target image;

FIG. 6E shows an example of display of diagnosis target image and information of representative disease cases;

FIG. 6F shows an example of a statistical information display screen;

FIG. 7A shows an example of a second-page retrieval result screen corresponding to FIG. 6E when the number of sample for a retrieved disease is small;

FIG. 7B shows an example of a similar disease case presentation screen including disorder information when the number of sample for the retrieved disease is small; and

FIG. 7C shows an example of a medical information presentation screen when the number of sample for the retrieved disease is small.

### DETAILED DESCRIPTION OF THE PREFERRED EMBODIMENTS

A preferred embodiment of an image diagnosis supporting apparatus and system according to the present invention will be described below with reference to the accompanying drawings.

### <System Configuration>

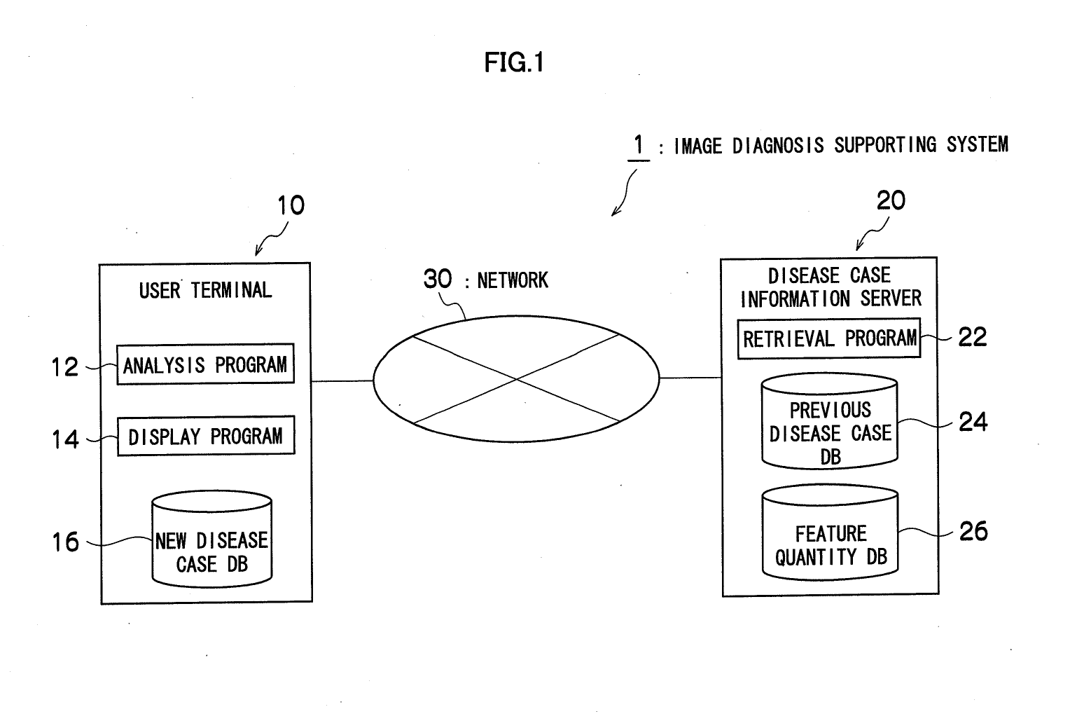

FIG. 1 is a system configuration diagram showing an image diagnosis supporting system according to an embodiment of the present invention.

This image diagnosis supporting apparatus 1 comprises a user terminal 10, a disease case information server 20, and a network 30 that connects the two.

The user terminal 10 is constituted by a personal computer or the like connected to the network 30, and primarily comprises an analysis program 12, a display program 14, an operating portion including a keyboard, a mouse and the like, a monitor, and a new disease case database (DB) 16.

The new disease case DB 16 stores images of new patients to be diagnosed (diagnosis targets). These images are captured by, for example, an imaging apparatus such as an X-ray CT apparatus, and are, for example, two-dimensional images including a lesion site or three-dimensional images including a lesion site.

A user uses the keyboard or the mouse to designate a two-dimensional image of a desired patient or a specific two-dimensional image on which a lesion site is captured from a three-dimensional image of the desired patient, and sets the two-dimensional image as a diagnosis target image (query image). The user terminal 10 is capable of displaying a two-dimensional image designated in this manner as a query image on the monitor.

The analysis program 12 calculates a feature quantity (first feature quantity) of a lesion site on the query image from the same. A plurality of types of feature quantities of a lesion site exists, such as shape, size, density or the like of the lesion site.

The display program 14 displays the aforementioned query image and results of an inquiry from the disease case information server 20 (disease case image, statistical information, diagnostic information, medical information or the like to be referenced) on the monitor.

The disease case information server 20 primarily comprises a retrieval program 22, a disease case DB 24 and a feature quantity DB 26.

The retrieval program 22 retrieves, in response to a retrieval request from the user, relevant information from the disease case DB 24 and the feature quantity DB 26, and transmits the retrieval results to the user terminal.

Disease case information including a disease case image to become a retrieval target image is classified according to disorder and stored in the disease case DB 24. Furthermore, with respect to each disorder, statistical information, disorder information and, with respect to particular disorders, disorder information and medical information of confusable disorders are registered in the disease case DB 24 with links provided.

Besides disease case images of each conclusively diagnosed disorder, disease case information includes text-based diagnostic information such as an interpretation report created by an interpreting doctor or the like.

Statistical information according to disorder includes, for example, information listed below.
A degree of similarity of a representative disease case (minimum degree of similarity within a disorder)
Average degree of similarity
Total number of registered disease cases of relevant disorders in the disease case DB
Number of registered disease cases (disease case patterns) similar to a representative disease case
Feature among disease case patterns—predominant symptom
Feature of patients among disease case patterns—average age, clinical history, smoking history and the like
Nationwide/local disease prevalence
Others

Feature quantities (second feature quantities) extracted from lesion sites of respective disease case images registered in the disease case DB 24 are classified according to disorder and stored in the feature quantity DB 26.

Each piece of disease case information by disorder that is stored in the disease case DB 24 and the second feature quantities corresponding to the respective pieces of disease case information stored in the feature quantity DB 26 are associated to each other by disease case IDs. Furthermore, the disease case DB 24 and the feature quantity DB may be arranged as a single DB.

### <Inputting Disease Case Information>

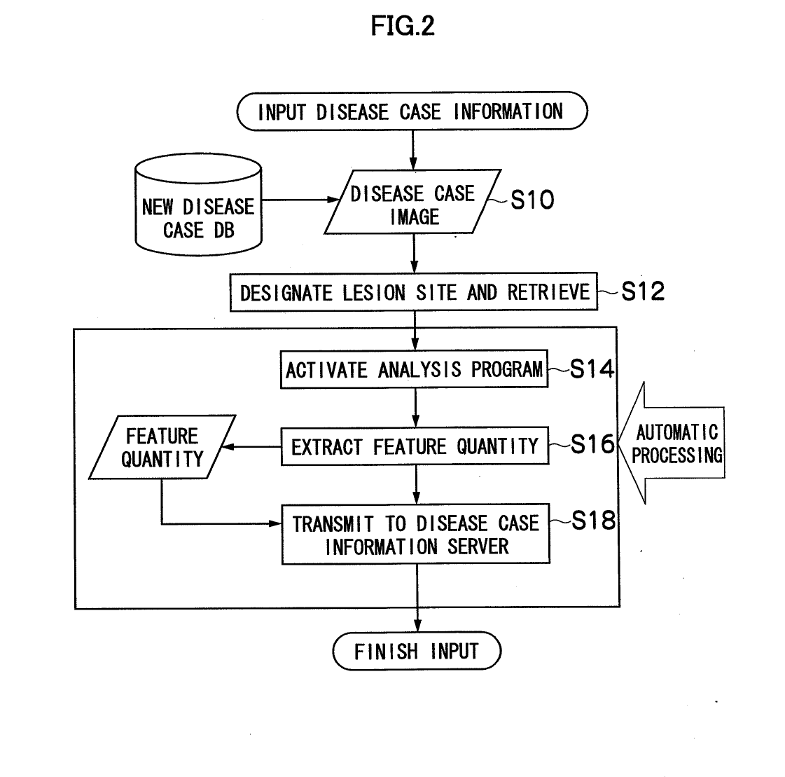

FIG. 2 is a flowchart showing processing performed on the user terminal side.

The user designates a diagnosis target image (disease case image) of a new patient from the new disease case DB 16 and causes the image to be displayed on the monitor of the user terminal 10 (step S10). The user then performs image diagnosis according to the disease case image displayed on the monitor, moves the mouse cursor to a lesion site on the disease case image, performs marking by one-click 3D measurement, and causes retrieval to be executed (step S12).

Upon receiving a retrieval instruction, an image of the lesion site is handed to the analysis program 12, whereby the analysis program 12 is activated (step S14). The analysis program 12 determines a feature quantity (first feature quantity) of the lesion site on the disease case image (step S16).

After extracting the first feature quantity, the first feature quantity is transmitted to the disease case information server 20 (step S18).

The disease case information server 20 retrieves disease case images which are the most similar to the diagnosis target image from the disease case DB 24 on a disorder-by-disorder basis based on the information.

### <Retrieving Disease Case Information>

The disease case information server 20 transmits the first feature quantity of the diagnosis target image (query image) received from the user terminal 10 to the retrieval program 22. The retrieval program 22 retrieves, on a disorder-by-disorder basis, disease cases with high degrees of similarity to the disease case of the query image from within the feature quantity DB 26 of previous disease cases. In the case where a relevant disease case exists, the disease case is used as a key to obtain detailed disease case information from the disease case DB 24.

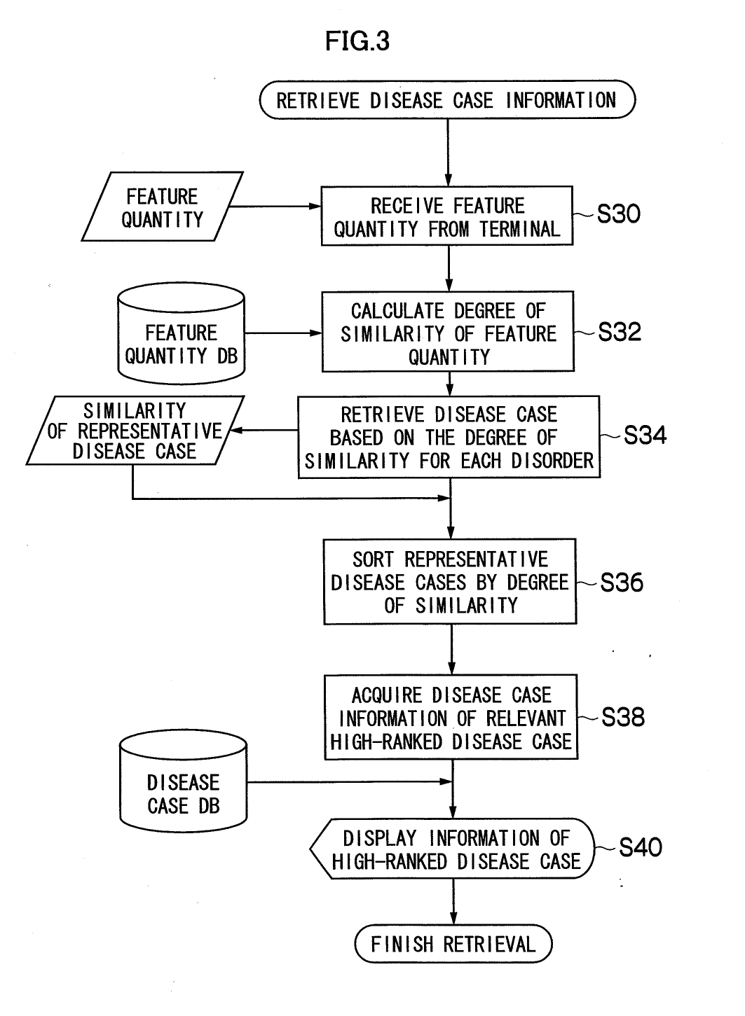

FIG. 3 is a flowchart showing processing performed on the disease case information server side.

The disease case information server 20 receives the first feature quantity of the query image from the user terminal 10 (step S30).

Next, the first feature quantity of the query image is compared with the second feature quantity of each disease case image classified by disorder in the feature quantity DB 26, and degrees of similarity between the two are calculated individually (step S32).

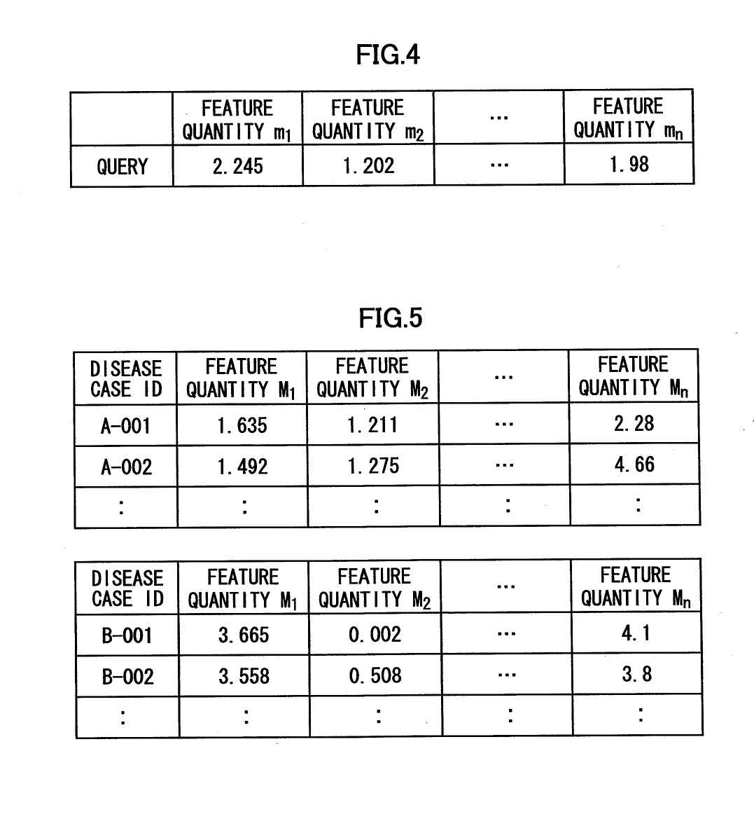

FIG. 4 shows a data example of first feature quantities extracted from a query image, and FIG. 5 shows data examples of second feature quantities of respective disease case images (A-001, A-002, . . . , B-001, B-002, . . . ) stored in the feature quantity DB 26 and classified by disorder (A, B, . . . ).

In step S32, based on a first feature quantity mᵢ (i=1, 2, . . . n) extracted from the query image and a second feature quantity Mᵢ (i=1, 2, . . . n) of each disease case image, a degree of similarity S between the query image and each disease case image is calculated according to the following formula.

$$S = {\sum }_{i = 1}^{n} {w}_{i} \vert {M}_{i} - {m}_{i} \vert \qquad [ Formula 1 ]$$

In Formula 1 presented above, wᵢ (i=1, 2, . . . , n) represents a weighting coefficient defined in advance on a disorder-by-disorder basis and applied to each feature quantity. As is apparent from Formula 1, the closer the calculated value of the degree of similarity S is to 0, the more similar the query image and the disease case image are to each other.

The above calculation of the degree of similarity S is performed separately on a disorder-by-disorder basis.

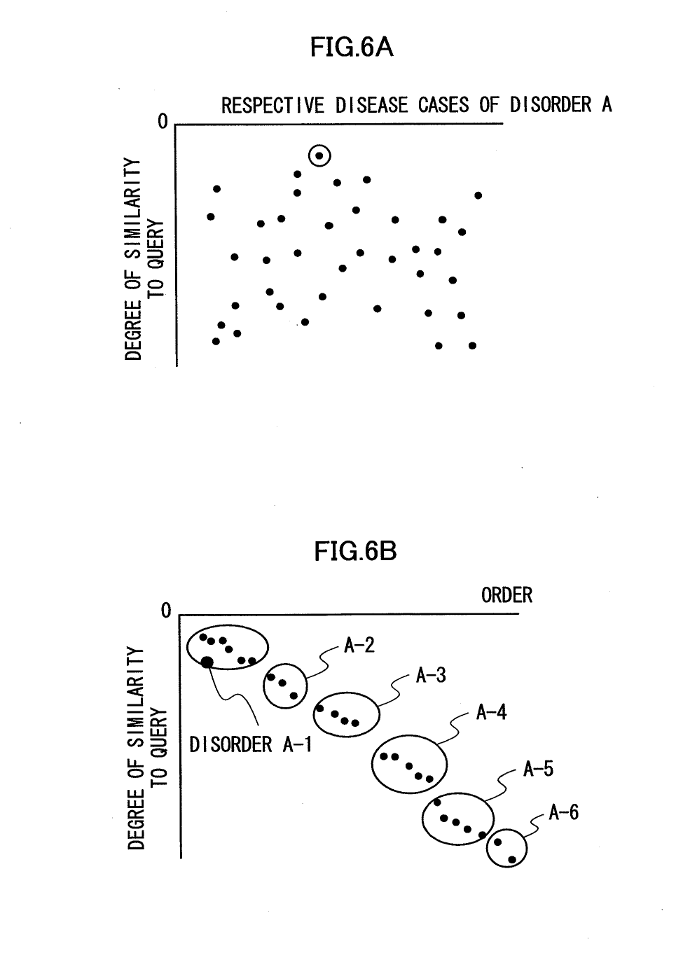

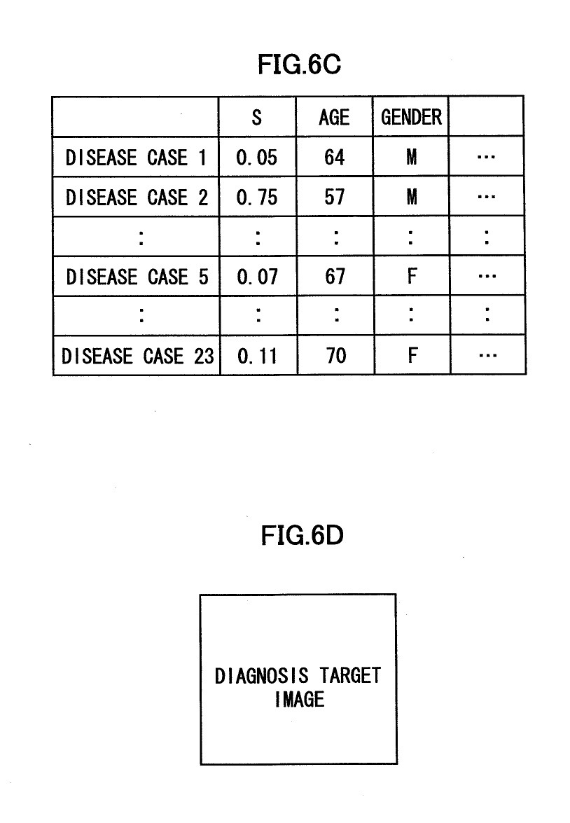

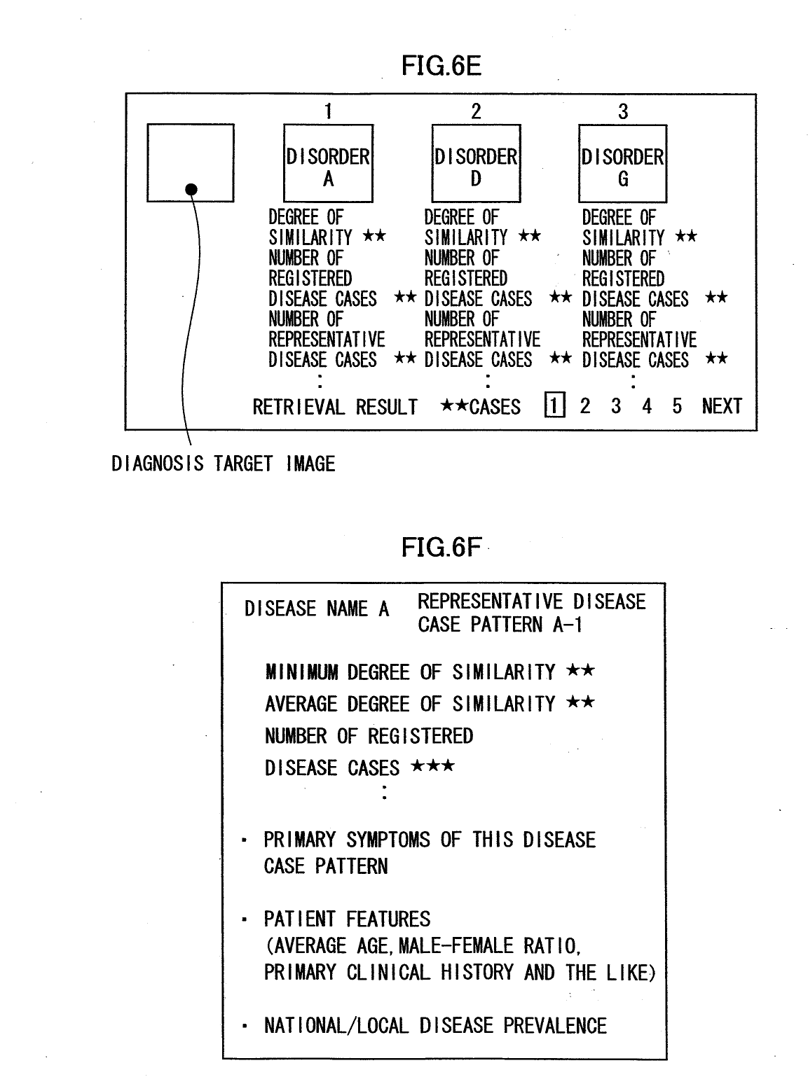

FIG. 6A is an example of a graph plotting a relationship between respective disease case images of a disorder A and a query image, where the abscissa represents respective disease cases and the ordinate represents degree of similarity. In the graph of FIG. 6A, the degree of similarity is 0 (zero) at the origin and the degree of similarity increases as a point goes downward with respect to the ordinate. That is, in FIG. 6, as a position of a point goes downward with respect to the ordinate, a disease case image corresponding to the point becomes less similar to the query image. In FIG. 6A, the encircled disease case image is the first most similar to the query image.

Returning now to FIG. 3, in step S34, disease case images which are the most similar to the query image (that is, images with the lowest degree of similarity S) (hereinafter referred to as representative disease cases) are retrieved on a disorder-by-disorder basis.

The representative disease cases by disorder which are retrieved in step S34 are sorted in an ascending order of degrees of similarity S (step S36).

Next, based on disease case IDs of the representative disease cases which are high-ranked in step S36 (high-ranked representative disease cases), corresponding disease case information is acquired from the disease case DB 24 (step S38). The high-ranked representative disease cases may be determined by selecting a predetermined number of disease case images among representative disease case sorted by the degree of similarity S or by selecting disease case images, each of which has a degree of similarity being less than a predetermined threshold value.

The disease case information server 20 transmits disease case information of the high-ranked representative disease cases acquired as described above to the user terminal 10, and causes the information to be displayed on the monitor of the user terminal 10 (step S40).

The present example has been arranged so that representative disease cases having the lowest degree of similarity S are retrieved on a disorder-by-disorder basis. However, even within the same disorder, there may be cases where image findings such as shape or density differ due to degrees of progression of the illness, etc., causing large variations in feature quantities (second feature quantities). In such a case, further classification (sub-classification) of disease case information may be performed in the same disorder.

Conceivable methods of performing sub-classification using degrees of similarity S or feature quantities include a method using an existing clustering method such as the nearest neighbor method or the k-means method, a method in which sub-classification is manually performed on disease cases registered in advance by a person, and the like. When using the clustering method, sub-classification may either be performed upon registration into the disease case DB or after retrieval based on the similarity.

FIG. 6B shows an example where respective retrieved disease case images of the disorder A are sorted according to degrees of similarity S and then clustered to classify them into sub-classifications (A-1, A-2, A-3, . . . ).

In the case where disease case images are sub-classified in the same disorder as described above, disease case information of representative disease cases is retrieved on a sub-classification by sub-classification basis, and then the disease case information is transmitted to the user terminal 10 to be displayed thereon.

### <Displaying Disease Case Information>

When representative disease cases are retrieved on a disorder-by-disorder basis through the above-described process, disease case information of each representative disease case is presented to the user.

FIGS. 6A to 6E are diagrams showing display examples of disease case retrieval images and retrieval results.

As shown in FIG. 6, representative disease cases are retrieved according to disorder based on the diagnosis target image (query image) (FIG. 6D), and the diagnosis target image and information of representative disease cases by disorder are displayed on the monitor of the user terminal 10 (FIG. 6E).

With the example shown in FIG. 6E, followings are displayed on a disorder-by-disorder basis: images of representative disease cases of disorders A, D and G, which are the first to third most similar to the diagnosis target image; degrees of similarity S with respect to the diagnosis target image, number of registered disease cases and numbers of representative disease cases. Further, number of retrievals (total number of retrieved disorders), a software button labeled “next page” for referencing information on other disorders that could not be displayed on a single screen, and the like are displayed.

The representative disease cases by disorder are, as described earlier, disease case images that are the first most similar to the query disease case image among the disease cases of the same disorder. For example, for the disorder A, the disease case image encircled in FIG. 6A is the representative disease case.

The number of representative disease cases by disorder corresponds to the number of clustered sub-classifications (A-1, A-2, A-3, . . . ), as shown in FIG. 6B.

In this case, when referencing statistical information of the disorder A, a retrieval result screen is switched to a statistical information display screen as shown in FIG. 6F by either selecting a representative disease case of the disorder A and pressing the enter key or by clicking a “statistical information” icon, not shown.

The statistical information displayed on the screen may be created on a disorder-by-disorder basis (or on a sub-classification by sub-classification basis). At this point, when displaying the statistical information of sub-classification A-1 of the disorder A, disease cases belonging to sub-classification A-1 are extracted from disease case data of the disorder A (disease case data of the disorder A is shown in FIG. 6C) and statistical information is calculated from the extracted disease case data.

The user is able to determine whether the disorder is a frequently occurring disorder or a rare disorder from statistical information of the disorder displayed as described above.

<Handling of Disease Cases with a Small Number of Samples>

With disorders with only a small number of disease case samples registered in the disease case DB, since large variation occurs in degrees of similarity S, the reliability of the degrees of similarity S is low. Therefore, for example, even with a disorder whose disease case image is determined to be very similar to the query image based on the degree of similarity, it is risky to accept the disorder at face value.

When such a rare disorder is retrieved, caution is raised about the disorder. More specifically, the disease case information server 20 comprises a determining device that determines whether a disorder is a rare disorder or not from the number of disease case samples of the disorder retrieved by the retrieval program 22 and an alarming device that attaches a warning message to rare disorders as will be described below.

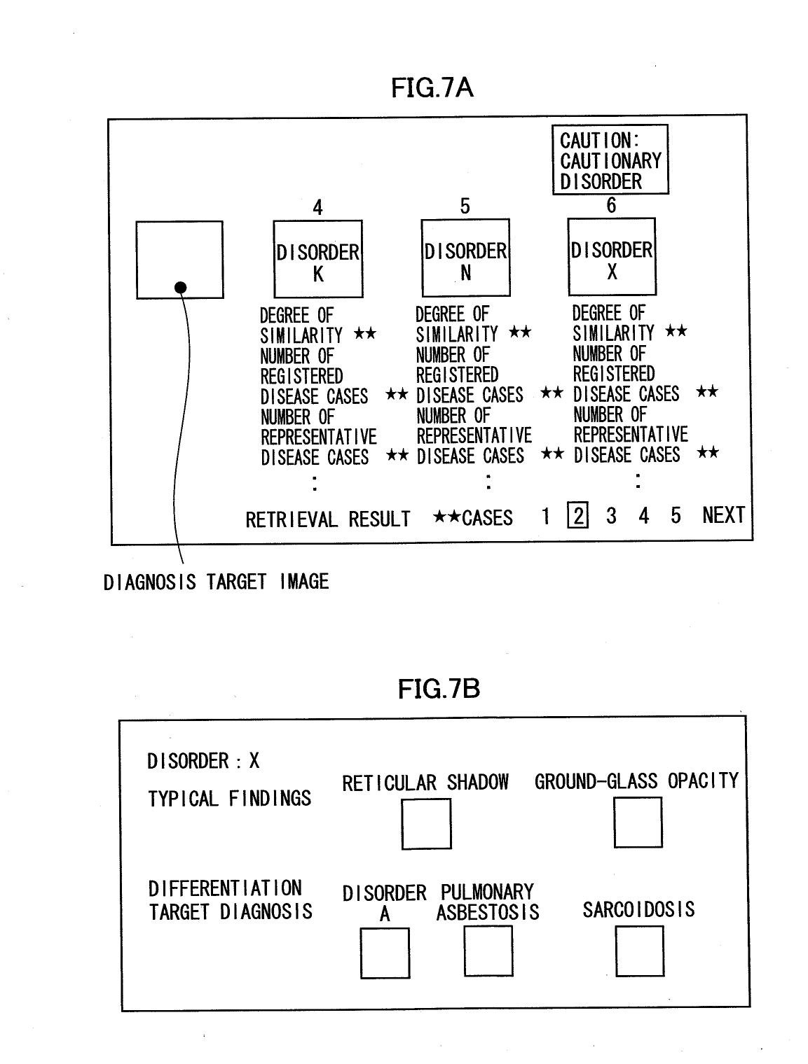

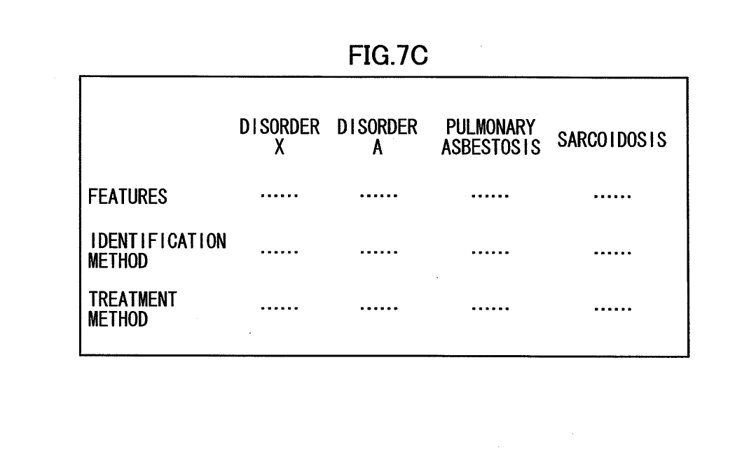

FIG. 7A is a second-page retrieval result screen corresponding to FIG. 6E, and displays images or the like of representative disease cases of three disorders K, N and X respectively of which disease case images are the fourth to sixth most similar to the query image.

In the example shown in FIG. 7A, the disorder X has a small number of disease case samples. Therefore, a warning message indicating that the disorder X is a “cautionary disorder” requiring attention during reference thereof is attached to the disorder X.

Due to the warning message “cautionary disorder”, the user is able to make a careful judgment by considering the fact that the number of samples is small when referencing disease case images or the like of the disorder X. In addition, with disorders attached with a “cautionary disorder” warning message, disorder information regarding the disorder, disorder information of confusable disorders and the like are arranged so as to be referable.

More specifically, a warning message is constituted as a link button, and by following the link a similar disease case presentation screen including disorder information (typical findings, images of lesion sites) of the disorder X and other disorder information on differentiation target disorders can be displayed as shown in FIG. 7B. By further following the link, as shown in FIG. 7C, a medical information presentation screen including medical information of the disorder X and the differentiation target disorder in a medical literature DB can be displayed.

With a disorder attached with a “cautionary disorder” warning message, the user can cause the above-described similar disease case presentation screen or the medical information presentation screen to be displayed and reference information displayed on these screens.

A warning display such as described above need not be limited to rare disease cases, and may be expanded so as to target disorders with a large number of disease cases but nevertheless require special attention.

### <Modification>

While the present embodiment is arranged so that the user terminal transmits a first feature quantity calculated from a query image to the disease case information server, the user terminal may be arranged to transmit the query image itself instead. Then, at the disease case information server 20 side, the feature quantity (first feature quantity) for retrieval is calculated based on the query image received from the user terminal.

In addition, the network 30 shown in FIG. 1 may be a secure external network such as IPSec, SSL-VPN, or the like, or an internal network such as an in-hospital network.

Furthermore, the present invention is not limited to an image diagnosis supporting system using a network and can also be applied to a self-contained image diagnosis supporting apparatus that internally performs all processing.

What is claimed is:

1. An image diagnosis supporting apparatus comprising:
a feature quantity calculating device which calculates, based on a diagnosis target image, a first feature quantity of a lesion site included in the diagnosis target image;
a database in which a plurality of disease case images and second feature quantities of lesion sites in the respective disease case images are classified by disorder and registered in association with each other;
a retrieving device which compares the first feature quantity with the second feature quantities on a disorder-by-disorder basis and retrieves a disease case image based on a comparison result on a disorder-by-disorder basis from the database; and
a displaying device which displays the disease case image retrieved by the retrieving device on a disorder-by-disorder basis.

2. The image diagnosis supporting apparatus according to claim 1, further comprising:
a determining device which determines whether a disorder requiring attention when reference thereof is included in the disease case images retrieved from the database; and
an alarming device which causes, when the determining device determines that the disorder requiring attention is included, a warning alerting that the disorder requiring attention is included to be displayed on the display device.

3. The image diagnosis supporting apparatus according to claim 2, wherein
the disorder requiring attention is a disorder for which a small number of samples is contained in the database.

4. The image diagnosis supporting apparatus according to claim 2, wherein
the alarming device causes at least one of disorder information regarding the disorder requiring attention and disorder information regarding confusable disorders to be displayed on the display device in association with the display of the warning.

5. The image diagnosis supporting apparatus according to claim 1, wherein
statistical information regarding disorders is registered into the database on a disorder-by-disorder basis;
the retrieving device retrieves disease case images and statistical information on a disorder-by-disorder basis from the database; and
the display device displays the disease case images and the statistical information retrieved by the retrieving device.

6. The image diagnosis supporting apparatus according to claim 1, wherein
the disease case images classified by disorder are further classified into sub-classifications within each disorder.

7. The image diagnosis supporting apparatus according to claim 6, wherein
the retrieving device compares the first feature quantity and the second feature quantities on a disorder-by-disorder basis, and retrieves a disease case image based on a comparison result on a disorder-by-disorder basis from the database and on a sub-classification by sub-classification basis.

8. An image diagnosis supporting system in which a user terminal and a disease case information server are connected to each other via a network, wherein
the user terminal comprises:
a feature quantity calculating device which calculates, based on a diagnosis target image, a first feature quantity of a lesion site included in the diagnosis target image;
a first communication device which transmits the calculated first feature quantity to the disease case information server and receives a disease case image retrieved by the disease case information server; and
a display device which displays the received disease case image, whereas
the disease case information server comprises:
a database in which a plurality of disease case images and second feature quantities of lesion sites in the respective disease case images are classified by disorder and registered in association with each other;
a retrieving device which compares the first feature quantity with the second feature quantities on a disorder-by-disorder basis and retrieves a disease case image based on a comparison result on a disorder-by-disorder basis from the database; and
a second communication device which receives the calculated first feature quantity from the user terminal and transmits the disease case image retrieved on a disorder-by-disorder basis to the user terminal.

9. The image diagnosis supporting system according to claim 8, wherein the disease case information server further comprises:
a determining device which determines whether a disorder requiring attention when reference thereof is included in the disease case images retrieved from the database; and
an alarming device which causes, when the determining device determines that the disorder requiring attention is included, a warning alerting that the disorder requiring attention is included to be displayed on the display device of the user terminal.

10. The image diagnosis supporting system according to claim 8, wherein:
statistical information regarding disorders is registered into the database on a disorder-by-disorder basis;
the retrieving device retrieves disease case images and statistical information on a disorder-by-disorder basis from the database; and
the second communication device transmits the disease case images and the statistical information retrieved by the retrieving device to the user terminal.

11. An image diagnosis supporting system in which a user terminal and a disease case information server are connected to each other via a network, wherein
the user terminal comprises:
a first communication device which transmits a diagnosis target image to the disease case information server and which receives a disease case image retrieved by the disease case information server; and
a display device which displays the received disease case image, whereas
the disease case information server comprises:
a feature quantity calculating device which calculates, based on the diagnosis target image, a first feature quantity of a lesion site included in the diagnosis target image;
a database in which a plurality of disease case images and second feature quantities of lesion sites in the respective disease case images are classified by disorder and registered in association with each other;
a retrieving device which compares the first feature quantity with the second feature quantities on a disorder-by-disorder basis and retrieves a disease case image based on a comparison result on a disorder-by-disorder basis from the database; and
a second communication device which receives the diagnosis target image from the user terminal and transmits the disease case image retrieved on a disorder-by-disorder basis to the user terminal.

12. The image diagnosis supporting system according to claim 11, wherein the disease case information server further comprises:
a determining device which determines whether a disorder requiring attention when reference thereof is included in the disease case images retrieved from the database; and
an alarming device which causes, when the determining device determines that the disorder requiring attention is included, a warning alerting that the disorder requiring attention is included to be displayed on the display device of the user terminal.

13. The image diagnosis supporting system according to claim 12, wherein
the alarming device causes at least one of disorder information regarding the disorder requiring attention and disorder information regarding confusable disorders to be displayed on the display device in association with the display of the warning.

14. The image diagnosis supporting system according to claim 11, wherein:
statistical information regarding disorders is registered into the database on a disorder-by-disorder basis;
the retrieving device retrieves disease case images and statistical information on a disorder-by-disorder basis from the database; and
the second communication device transmits the disease case images and the statistical information retrieved by the retrieving device to the user terminal.
